(19) United States
(12) Patent Application Publication
(10) Pub. No.: US 2006/0064056 A1
(43) Pub. Date: Mar. 23, 2006

(54) Guiding catheter assembly for embolic protection by proximal occlusion

(76) Inventors: James Coyle, Somerville, MA (US); Raymond E. Godaire, Auburn, MA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/944,312

(22) Filed: Sep. 17, 2004

(51) Int. Cl. A61M 29/00

(52) U.S. Cl. 604/96.01

(57) ABSTRACT

A guiding catheter assembly and a method for using the assembly for treating a stenotic lesion. The guiding catheter assembly includes a guiding catheter having an occluder balloon mounted distally thereon. A flexible leader is slidably disposed through a primary lumen of the guiding catheter and has a tapered distal portion extending distally of the guiding catheter. The guiding catheter may have a preformed curve at the distal end. An embodiment of the guiding catheter may also comprise at least one steering wire disposed within a steering lumen in the guiding catheter, the steering wire being operable to deflect the distal end of the guiding catheter.

### FIELD OF THE INVENTION

The present invention relates generally to intraluminal devices, and more particularly, to a guiding catheter assembly for advancing a medical device through a patient's vasculature.

### BACKGROUND OF THE INVENTION

Stenotic lesions form on the lumen walls of a blood vessel to create narrowings that restrict blood flow there through, and may comprise a hard, calcified substance and/or a softer thrombus material. Interventional catheterization procedures such as balloon angioplasty, stent deployment, atherectomy, and thrombectomy are well known and have proven effective in the treatment of such stenotic lesions. Such modalities require the insertion of a therapy catheter through a patient's vasculature.

Recently, devices have been developed that address concerns relating to atheroembolization; i.e. the obstruction of blood vessels by stenotic debris that may be released during interventional catheterization therapies such as those mentioned above. Distal protection devices (DPDs) such as filters and occluders represent one class of intravascular devices that can be used to prevent atheroembolization. A filter mounted on a guidewire or a catheter may be positioned distally of a stenotic lesion to capture and remove embolic debris without causing hemostasis. Alternatively, an occluder guidewire or catheter may be positioned distally of a stenotic lesion to temporarily stop the flow of blood and any stenotic debris that may have become entrained in the blood. The contaminated blood is aspirated from the treated area before the occluder device is collapsed to permit resumption of blood flow.

Occlusion devices may also be placed proximally of a stenotic lesion to provide proximal protection. Such proximal occluders may be used alone to prevent atheroembolization, or may be used in conjunction with a distal occluder to form an isolated treatment chamber about the lesion to be treated. Preliminary deployment of a proximal occlusion device may be advantageous in preventing atheroembolization because advancing a treatment catheter into a tight stenosis can dislodge particulate debris, even before the stenosis is opened.

A guiding catheter is typically used to direct a treatment catheter to a stenotic lesion, especially if the lesion is remote from the vascular assess site. One type of guiding catheter that may be utilized is described in U.S. patent appn. No. 2002/0026145 A1 entitled “Method and Apparatus for Emboli Containment” to Bagaoisan et al. (“Bagaoisan”). Typical of most guiding catheters, the Bagaoisan catheter is pre-curved at the distal end to set and hold a supporting position in the vasculature while the therapeutic catheter crosses and treats the lesion. Additionally, the Bagaoisan catheter includes an expandable sealing balloon disposed around the guiding catheter's distal end that, when appropriately positioned, may be inflated to provide embolic protection by proximal occlusion. However, the Bagaoisan catheter may present certain drawbacks. For example, insertion of the sealing balloon for proximal occlusion requires deeply intubating the catheter tip into the selected vessel. Ordinarily, deep intubation with a guiding catheter is to be avoided to prevent scraping injury to the intimal lining of the vessel wall. Furthermore, a catheter that is designed for deep intubation and occlusion has a preformed curve with a tip portion that is longer than a similar curve not intended for deep intubation. With such an extended tip portion, it may be difficult to advance the guiding catheter through the patient's vasculature.

It would therefore be desirable to provide a guiding catheter capable of deep intubation within a patient's vasculature with minimal trauma to the patient's vessels. It would further be desirable to provide a device for use within a guiding catheter to ease advancement of the guiding catheter through the patient's vasculature. It would also be desirable to provide a guiding catheter capable of being steered during insertion by the user. Other desirable features and characteristics of the present invention will become apparent from the subsequent detailed description and the appended claims taken in conjunction with the accompanying drawings.

### BRIEF SUMMARY OF THE INVENTION

In one exemplary embodiment, a guiding catheter assembly is provided that includes a guiding catheter, a balloon, and a flexible leader. The guiding catheter has a distal end and a proximal end and has a primary lumen and an inflation lumen that extend there through. The inflation lumen has a proximal port, and the balloon is mounted adjacent the distal end of the guiding catheter and is in fluid communication with the proximal port. The flexible leader is configured to be slidably disposed within the primary lumen of the guiding catheter and has a tapered distal portion.

In another exemplary embodiment of the invention, a guiding catheter assembly is provided that includes a guiding catheter, a steering wire, a balloon, and a flexible leader. The guiding catheter has distal and proximal ends, and has a primary lumen, a steering lumen, and an inflation lumen each extending there through. The steering wire is disposed within the steering lumen and has a distal end attached to the distal end of the guiding catheter. The inflation lumen has a proximal port, and the balloon is mounted adjacent the distal end of the guiding catheter and is in fluid communication with the proximal port. The flexible leader is configured to be slidably disposed within the primary lumen and has a tapered distal portion.

In yet another exemplary embodiment, a guiding catheter assembly is provided including a guiding catheter, a steering wire, and a balloon. The guiding catheter has distal and proximal ends, and a primary lumen, a steering lumen, and an inflation lumen each extending there through. The steering wire is disposed within the steering lumen and has a distal end coupled to the distal end of the guiding catheter. The inflation lumen has a proximal port, and the balloon is mounted adjacent the distal end of the guiding catheter and is in fluid communication with the proximal port.

In still another exemplary embodiment, a method is provided for treating a stenotic lesion in a vessel of a patient. The method includes providing a guiding catheter assembly comprising a guiding catheter having an occlusion balloon mounted thereon and a flexible leader extending there through, advancing the guiding catheter assembly through the vessel to a location adjacent the stenosis, inflating the balloon to occlude the vessel, and withdrawing the flexible leader from the guiding catheter.

### BRIEF DESCRIPTION OF THE DRAWINGS

The following drawings are illustrative of a particular embodiment of the invention and therefore do not limit the scope of the invention. They are presented to assist in providing a proper understanding of the invention. The drawings are not to scale and are intended for use in conjunction with the explanations in the following detailed description. The present invention will hereinafter be described in conjunction with the appended drawings, wherein like reference numerals denote like elements, and:

FIG. 1 is a longitudinal cross-sectional view of an exemplary guiding catheter assembly in accordance with the invention;

FIG. 2 is a cross-sectional view taken along line 2-2 of the exemplary guiding catheter assembly illustrated in FIG. 1;

FIG. 3 is a longitudinal cross-sectional view of the guiding catheter shown in FIG. 1;

FIG. 4 is a longitudinal cross-sectional view of the flexible leader shown in FIG. 1; and

FIGS. 5-11 illustrate the use of the guiding catheter assembly shown in FIGS. 1 and 2 during a typical angioplasty procedure.

### DETAILED DESCRIPTION OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary, or the following detailed description.

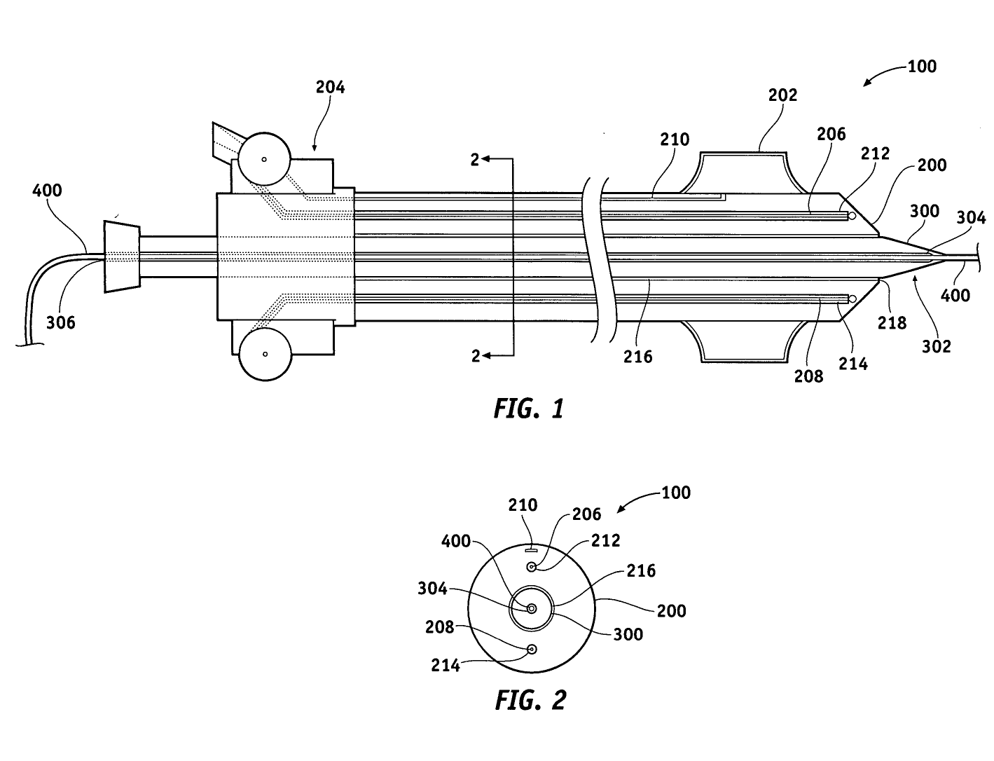

Referring to FIGS. 1 and 2, guiding catheter assembly 100 is provided according to an exemplary embodiment of the present invention. Guiding catheter assembly 100 comprises guiding catheter 200, flexible leader 300, and guidewire 400, each of which will be discussed in more detail below. Occlusion balloon 202 is mounted adjacent the distal end of catheter 200, and hub 204 is coupled to the proximal end of catheter 200. Occlusion balloon 202 and hub 204 are in fluid communication via inflation lumen 210. Steering wires 206 and 208 are disposed within steering lumens 212 and 214, respectively, that extend between the distal and proximal ends of guiding catheter 200. Guiding catheter 200 further comprises primary lumen 216 that also extends between the distal and proximal ends of guiding catheter 200. Flexible leader 300, configured for slidable movement within primary lumen 216, includes tapered distal portion 302 that is configured to protrude out of guiding catheter 200 through distal port 218. Flexible leader 300 is provided with wire lumen 304 for slidably receiving guidewire 400 there through.

Guiding catheter 200 is configured to have sufficient structural integrity to advance through a patient's vasculature to remote arterial locations without buckling. In this regard, guiding catheter 200 may be constructed of one or more flexible biocompatible materials, including, but not limited to, polyethylene, polypropylene, polyurethane, polyesters, or PEBAX® polyethylene block amide copolymer, available from ELF Atochem, Philadelphia, Pa., U.S.A.

If desired, a layer of braided filaments that resists kinking and enhances longitudinal transmission of rotation may be embedded within guiding catheter 200. Furthermore, the stiffness of guiding catheter 200 may be varied along the length thereof. For example, the distal end of guiding catheter 200 may possess physical properties that are different from those associated with the catheter's proximal end; e.g. guiding catheter 200 may become increasingly flexible in a distal direction.

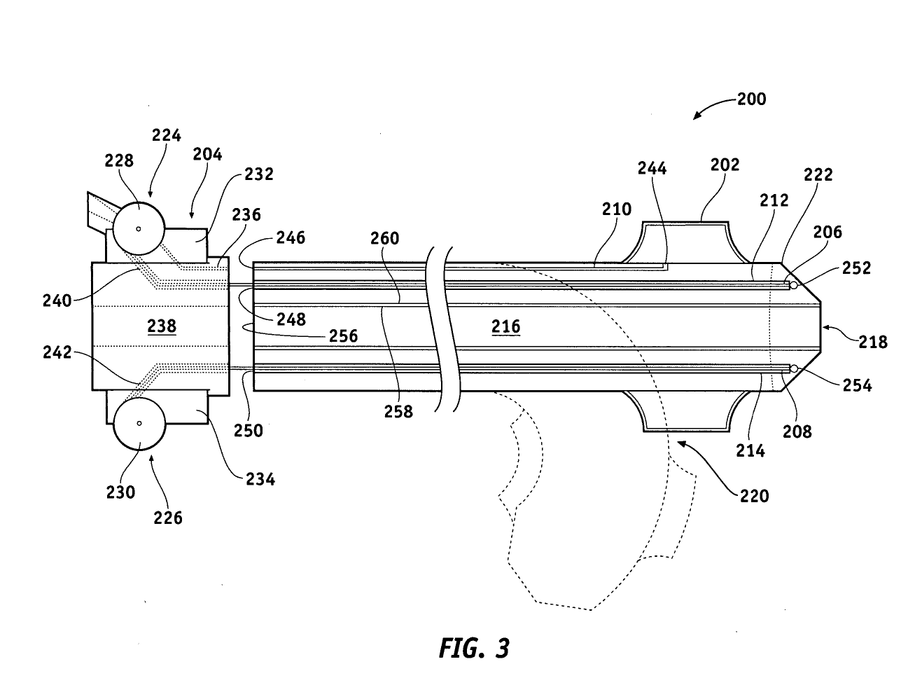

FIG. 3 is a longitudinal cross-sectional view of guiding catheter 200. Guiding catheter 200 may be substantially straight, or guiding catheter 200 may include curve 220, shown in phantom, proximate the distal region of guiding catheter 200. Any one of a number of pre-formed curve shapes may be incorporated into guiding catheter 200, such as Judkins-type or Amplatz-type curves, as non-limiting examples. Curve 220 may be pre-formed utilizing various known methods including, but not limited to, the method disclosed in U.S. Pat. No. 5,902,287 entitled “Guiding Catheter and Method of Making Same.” A desired curve 220 may be manually created from a straight or pre-formed distal region of guiding catheter 200 by manipulation of one or more steering wires, as will be described more fully below.

The distal end of guiding catheter 200 is configured to provide safe advancement of the catheter through a patient's vasculature. In one exemplary embodiment, the distal end of guiding catheter 200 is smooth and blunt. In another embodiment, the distal end is rounded. In yet another exemplary embodiment, soft distal segment 222 (shown in phantom) is formed of a soft material and coupled to the distal end of guiding catheter 200.

Occlusion balloon 202 is mounted to guiding catheter 200 utilizing any suitable technique known in the art (e.g. adhesive or heat bonding). Occlusion balloon 202 is configured to inflate and expand to a diameter sufficient to occlude a vessel within which it is positioned. The balloon may be formed of an elastic material, such as styrene-ethylene-butylene-styrene (SEBS), silicone, or latex. Alternatively, occlusion balloon 202 may be made of an inelastic, flexible biocompatible polymer.

Hub 204 is coupled to the proximal end of guiding catheter 200 and may be made of a hard polymer (e.g. medical grade polycarbonate, polyvinyl chloride, acrylic, acrylonitrile butadiene styrene (ABS), or nylon) or a metal that possesses the requisite structural integrity to provide a functional access port to guiding catheter 200; i.e. for balloon inflation or fluid aspiration. Hub 204 further comprises steering mechanisms 224 and 226 including actuators 228 and 230, respectively (e.g. reels). Steering mechanisms 224 and 226 are preferably at least partially disposed within housings 232 and 234, respectively.

Hub 204 is provided with balloon inflation lumen 236, device lumen 238, and at least first and second steering wire lumens 240 and 242. Each of lumens 236, 238, 240, and 242 adjoins a corresponding guiding catheter lumen 210, 216, 212, and 214, respectively, each of which will be discussed in more detail below.

Inflation lumen 210 carries fluid to and from occlusion balloon 202 to inflate and deflate occlusion balloon 202, respectively. In this regard, inflation lumen 210 extends substantially the length of guiding catheter 200 and includes distal balloon inflation port 244 and proximal fluid supply port 246. Preferably, inflation lumen 210 is formed adjacent the outer periphery of guiding catheter 200. Although a single inflation lumen 210 is depicted in FIG. 3, it will be appreciated that additional inflation lumens may be employed.

Steering lumens 212 and 214 each extend substantially the length of guiding catheter 200 and include wire entry ports 248 and 250, respectively, at the proximal end of guiding catheter 200. Each steering lumen 212 and 214 terminates within guiding catheter 200 adjacent its distal end and has a diameter sufficient to permit steering wires 206 and 208 respectively to slidably move therein. In the embodiment depicted in FIGS. 1-3, steering lumens 212 and 214 are substantially diametrically opposed; however; those with skill in the art will appreciate that this is not a requirement. Additionally, although two steering lumens are shown, it will be appreciated that the number of steering lumens may be varied to suit particular needs and/or applications.

Steering wires 206 and 208 are disposed within steering lumens 212 and 214, respectively. The steering wires may be comprised of a variety of materials having sufficient strength to bend guiding catheter 200 when pulled. In this regard, steering wires 206 and 208 are attached adjacent the distal end of guiding catheter 200 in one of a number of ways. In the exemplary embodiment shown in FIG. 3, the distal end of each steering wire 206 and 208 is anchored adjacent the distal end of the catheter as is shown at 252 and 254 respectively. The proximal ends of each steering wires 206 and 208 are coupled to actuators 228 and 230, respectively. Rotating the actuators causes steering wires 206 and/or 208 to pull at the distal end of guiding catheter 200 to deflect the distal end of guiding catheter 200 (e.g. create curve 220). That is, as configured in FIG. 3, pulling on wire 208 will cause the distal end of catheter to bend downward (shown in phantom). Pulling on wire 206 will cause an upward deflection.

Those with skill in the art may appreciate that actuators 228 and 230 may take other forms, such as manually operable slides to push/pull on steering wires 206 and 208. Skilled artisans will also recognize that the terms “steer” and “steering,” as used herein generally refer to various wires, lumens and actions causing deflection of the distal end of guiding catheter 200. However, it is to be understood that steering of guiding catheter 200 may include manual rotation of the catheter proximal end to cause rotation of the catheter distal end, with or without actions to cause simultaneous deflection of the distal end.

As noted above, guiding catheter 200 is provided with primary lumen 216 including inlet port 256 and distal port 218. Primary lumen 216 may include slippery interior surface 258 for reducing frictional forces between the interior surface 258 and devices that may be moved through primary lumen 216. In one exemplary embodiment, interior surface 258 is provided with a slippery coating, such as a silicone compound or a hydrophilic polymer. In another exemplary embodiment, interior surface 258 includes liner 260 formed from a slippery material that is coupled to interior surface 258. Those with skill in the art may appreciate that any one of numerous low-friction, biocompatible materials such as, for example, fluoropolymers (e.g. PTFE, FEP), polyolefins (e.g. polypropylene, high-density polyethylene), or polyamides, may be used to make interior surface 258.

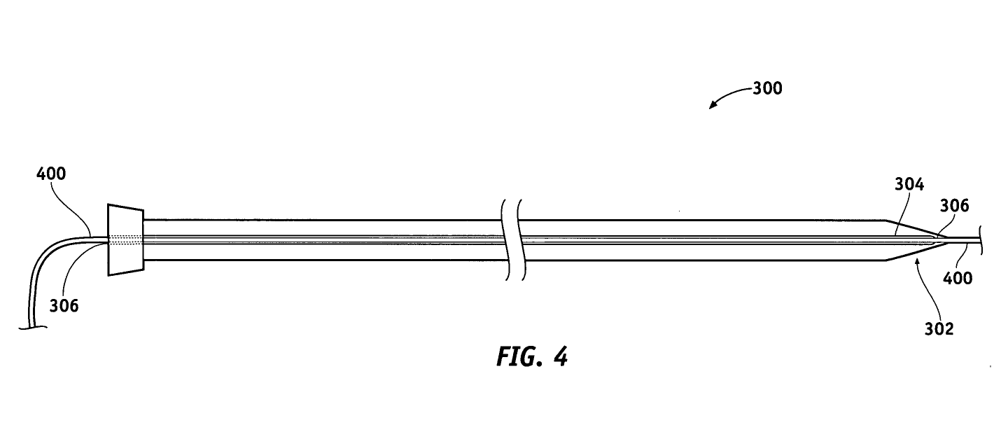

With reference to FIG. 4, flexible leader 300 may be inserted into primary lumen 216 and has tapered distal portion 302 that is configured to protrude from distal port 218 as shown in FIG. 1. Flexible leader 300 may be constructed of any suitable biocompatible material or combinations of materials, including, but not limited to, a fluoropolymer, a polyolefin, a polyurethane, or PEBAX, and may have either a stiff or flexible construction. Distal portion 302 is provided with tip 306, which is formed of relatively soft material to minimize trauma to the patient's vessels. Tip 306 may be closed and rounded, or tip 306 may have a distal opening to wire lumen 304, which will be described below. The outer diameter of flexible leader 300 may have a close sliding fit with guiding catheter 200 to substantially fill primary lumen 216. Alternatively, a distal region of flexible leader 300 may have a close sliding fit within distal port 218, while the remaining portion of flexible leader 300 is smaller in diameter to provide greater clearance through primary lumen 216. Distal portion 302 provides a tapered transition between tip 306 and guiding catheter 200, and serves as a leading surface when guiding catheter assembly 100 is advanced through a patient's vasculature.

Flexible leader 300 is further provided with wire lumen 304 that has a diameter sufficient to allow guidewire 400 to slidably move there through. As will be appreciated by those with skill in the art, the diameter of wire lumen 304 may or may not be uniform along its entire length. For example, in one alternative embodiment (not shown) of the flexible leader, the wire lumen proximal to the distal tip is larger in diameter than the opening in the distal tip to allow reduced wall thickness, which may result in an increase in flexibility along the length of flexible leader 300.

Guidewire 400 is used to guide medical devices such as guiding catheter 200 and/or therapeutic catheters to a desired position within the patient's vasculature. Guidewire 400 is conventional, having sufficient flexibility, especially at a distal end, to pass safely through a patient's vasculature, while having sufficient stiffness to partially straighten a pre-formed curve 220 of guiding catheter 300.

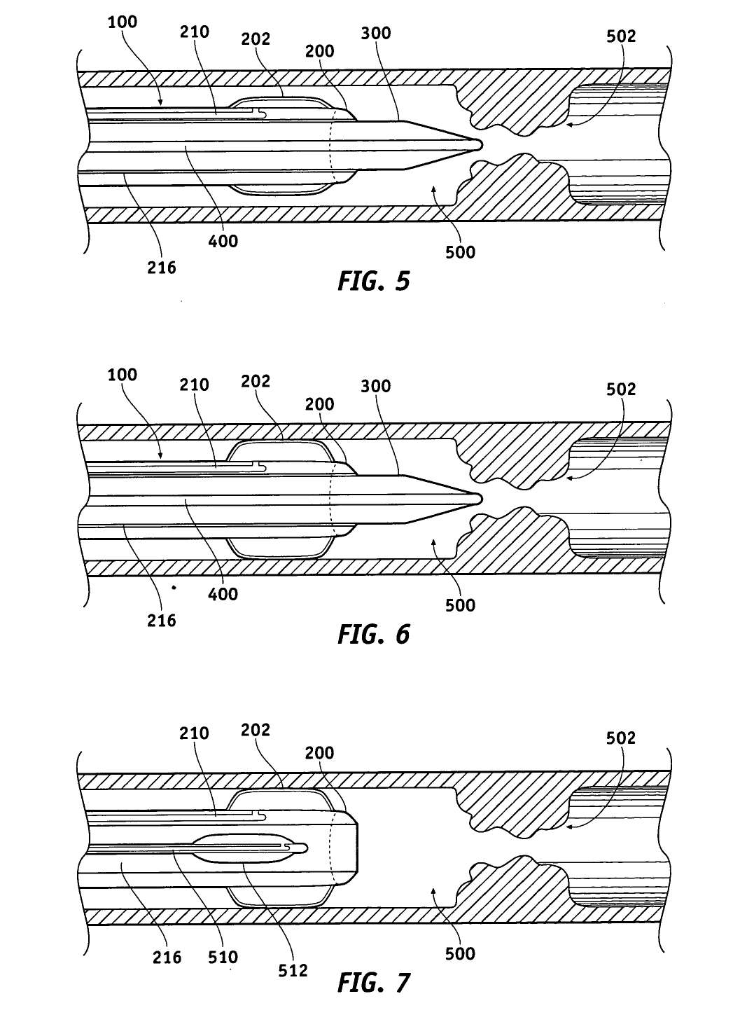

FIGS. 5-11 illustrate a method for the operation and use of guiding catheter assembly 100. Guiding catheter assembly 100 is advanced into vessel lumen 500 until occlusion balloon 202 is positioned between the ostium (not shown) and stenosis 502 to be treated, as illustrated in FIG. 5. In one exemplary embodiment of the method, guidewire 400 is first advanced to the desired position and guiding catheter 200 and flexible leader 300 are advanced over guidewire 400. In another exemplary embodiment, guiding catheter 200, flexible leader 300, and guidewire 400 are advanced to the desired position as a single unit. It will be recalled from above that a practitioner may use steering wires 206 and 208 (FIGS. 1 and 2) to deflect the distal end of guiding catheter 200 to aid the practitioner in the advancement of guiding catheter assembly 100 through a patient's vasculature. After being properly positioned within vessel lumen 500, occlusion balloon 202 is inflated until it engages the sidewall of the vessel as shown in FIG. 6. Flexible leader 300 and/or guidewire 400 may be retracted from primary lumen 216 before or after inflation of occlusion balloon 202.

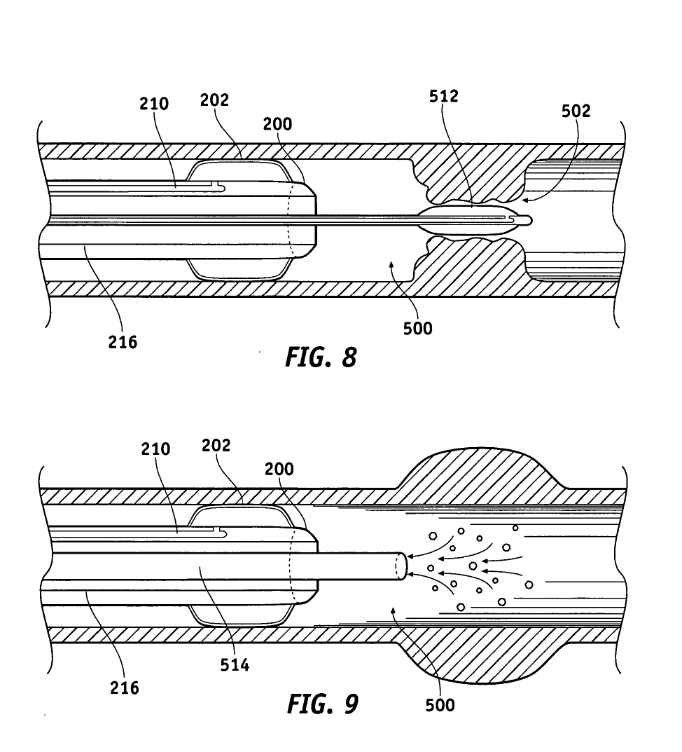

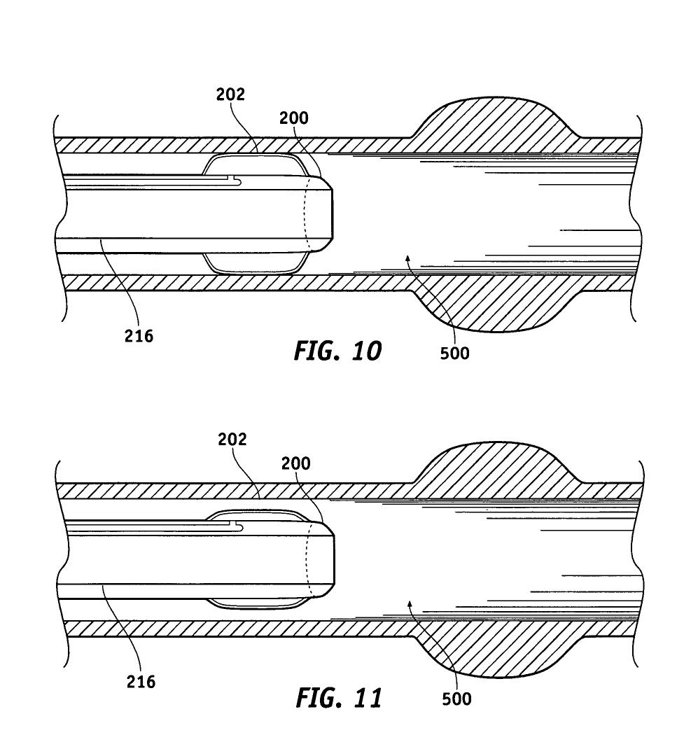

FIGS. 7-8 illustrate an exemplary procedure wherein balloon dilatation catheter 510, including dilatation balloon 512, is advanced through primary lumen 216 until the balloon reaches a desired position within stenosis 502. Dilatation balloon 512 is then inflated to dilate the stenosis. Balloon dilatation catheter 510 may then be removed, and any debris present may be aspirated, either directly into guiding catheter distal port 218 or, alternatively, into aspiration catheter 514, which may be advanced to the treated area within vessel lumen 500, as shown in FIG. 9. If used, aspiration catheter 514 may be subsequently withdrawn from primary lumen 216, as shown in FIG. 10. Then, occlusion balloon 202 is deflated as shown in FIG. 11, allowing blood flow to resume. Lastly, guiding catheter 200 is withdrawn from vessel lumen 500.

Thus, there has been provided a guiding catheter assembly configured for deep intubation within a patient's vasculature with minimal trauma to the patient's vessels. Furthermore, the guiding catheter assembly provides a smooth transition between the guiding catheter's distal end and the patient's vasculature to ease advancement of the guiding catheter through the patient's vasculature. The device is, if desired, also capable of being steered by the operator during insertion.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A guiding catheter assembly configured to receive a medical device there through, the assembly comprising:
a guiding catheter comprising:
an elongate shaft having proximal and distal ends and a pre-formed, curved distal region;
a primary lumen and an inflation lumen extending through the shaft, the primary lumen terminating in a distal port at the shaft distal end;
a balloon mounted adjacent the shaft distal end and being in fluid communication through the inflation lumen with a fluid supply port at the shaft proximal end; and
an elongate flexible leader configured to have a close, sliding fit within the primary lumen such that a tapered distal portion of the flexible leader extends distally from the guiding catheter distal port.

2. The assembly of claim 1, wherein the flexible leader further comprises a wire lumen for slidably receiving a guidewire there through.

3. The assembly of claim 2, wherein the tapered distal portion of the flexible leader presents a smooth transition from the guidewire to the shaft distal end.

4. The assembly of claim 1, wherein the flexible leader has a substantially smooth, rounded distal tip.

5. The assembly of claim 1, wherein the guiding catheter further comprises:
a first steering lumen extending there through; and
a first steering wire having a distal end and a proximal end, the first steering wire disposed within the first steering lumen and the distal end of the first steering wire attached to the distal end of the shaft.

6. The assembly of claim 5, wherein the guiding catheter further comprises:
a second steering lumen extending there through; and
a second steering wire having a distal end and a proximal end, the second steering wire disposed within the second steering lumen and the distal end of the second steering wire attached to the distal end of the shaft.

7. The assembly of claim 5, further comprising:
a hub coupled to the proximal end of the shaft, the hub comprising an actuator coupled to the proximal end of the first steering wire.

8. The assembly of claim 1, wherein the stiffness of at least a first portion of the shaft is different from the stiffness of at least a second portion of the shaft.

9. The assembly of claim 1, wherein the distal end of the shaft is substantially smooth and blunt.

10. The assembly of claim 1, further comprising a liner formed of a slippery material, wherein the primary lumen includes a surface to which the liner is coupled.

11. A guiding catheter assembly configured to receive a medical device there through, the assembly comprising:
a guiding catheter comprising:
an elongate shaft having distal and proximal ends;
a primary lumen and a steering lumen extending through the shaft, the primary lumen terminating in a distal port at the shaft distal end;
an inflation lumen extending from a fluid supply port at the shaft proximal end to an inflation port adjacent the shaft distal end;
a steering wire disposed within the steering lumen, the steering wire having a distal end attached to the distal end of the shaft;
a balloon mounted adjacent the shaft distal end and being in fluid communication with the fluid supply port through the inflation lumen; and
a flexible leader configured to have a close, sliding fit within the primary lumen such that a tapered distal portion of the leader extends distally from the shaft.

12. The assembly of claim 11, wherein the flexible leader further comprises a wire lumen for slidably receiving a guidewire there through.

13. The assembly of claim 1 1, wherein the shaft further comprises a pre-formed curved distal region.

14. A guiding catheter configured to receive a medical device there through, the catheter comprising:
an elongate shaft having proximal and distal ends and a pre-formed, curved distal region;
a primary lumen extending through the shaft and terminating in a distal port at the shaft distal end;
an inflation lumen extending from a fluid supply port at the shaft proximal end to an inflation port adjacent the shaft distal end;
a steering lumen extending through the shaft;
a steering wire slidably disposed within the steering lumen, the steering wire having a distal end attached to the distal end of the shaft; and
a balloon mounted adjacent the shaft distal end and being in fluid communication with the fluid supply port through the inflation lumen.

15. The catheter of claim 14, further comprising a flexible leader configured to have a close, sliding fit within the primary lumen such that a tapered distal portion of the leader extends distally from the shaft.

16. A method for treating a stenotic lesion in a vessel of a patient, the method comprising:
providing a guiding catheter assembly comprising a guiding catheter having an occlusion balloon mounted thereon and a flexible leader extending there through;
advancing the guiding catheter assembly in the patient until the balloon is positioned in the vessel at a location proximal to the lesion;
inflating the balloon to occlude the vessel; and
withdrawing the flexible leader from the guiding catheter.

17. The method of claim 16, further comprising advancing a treatment catheter through the guiding catheter.

18. The method of claim 16, further comprising advancing an aspiration catheter through the guiding catheter.

19. The method of claim 16, further comprising pulling a steering wire disposed within the catheter to deflect the distal end of the guiding catheter assembly.

20. The method of claim 16, further comprising deflating the balloon and removing the guiding catheter from the patient.
